(19) United States
(12) Patent Application Publication
(10) Pub. No.: US 2007/0243556 A1
(43) Pub. Date: Oct. 18, 2007

(54) Smart combinatorial operando spectroscopy catalytic system

(75) Inventor: Israel E. Wachs, Bridgewater, NJ (US)

Correspondence Address: BANNER & WITCOFF, 1001 G STREET N W, SUITE 1100, WASHINGTON, DC, 20001, US

(73) Assignee: Catalyst Design Inc, Bridgewater, NJ (US)

(21) Appl. No.: 11/098,775

(22) Filed: Apr. 14, 2005

Related U.S. Application Data
(60) Provisional application No. 60/561,880, filed on Apr. 14, 2004.

(51) Int. Cl. G01N 33/53; C12M 1/34; G01N 33/558; G01N 33/543

(52) U.S. Cl. 435/7.1; 435/287.1; 436/514; 436/518

(57) ABSTRACT

A device and combinatorial method is disclosed for screening a plurality of catalytic materials simultaneously while determining the dynamic bulk and surface nature of the catalytic materials being screened under reaction conditions and surface chemical kinetic and mechanistic information for determining the structure-activity/selectivity relationship of the catalytic materials, and for collecting information on the dynamic structures of the catalytic materials as well as surface species. The discovery process of novel materials may thereby be accelerated, the associated costs may be reduced, and the information may also lead to the design of improved and advanced materials.

This application claims priority to U.S. Provisional Patent Application Ser. No. 60/561,880, filed Apr. 14, 2004, the entire contents of which are hereby incorporated by reference herein.

### FIELD OF THE INVENTION

Aspects of the present invention are directed to materials research and development as well as spectroscopy.

### BACKGROUND OF THE INVENTION

Materials research encompasses an unusually broad range of different materials including organic and inorganic materials, biomaterials, pharmaceutical materials, food materials, nanomaterials, photonic materials, catalytic materials and functional materials. These materials find wide application as sensors for process control, transmission of data, catalytic materials for environmental, chemical and petroleum industries applications, stronger and lighter structural materials, artificial human body parts, and novel drug delivery systems.

The acceleration of the discovery of new materials and novel properties also has many social benefits. For example, catalytic materials are currently employed throughout the petroleum and chemical industry to manufacture various products such as fuels, polymers, chemicals, and textile fibers. The discovery of new, more efficient and novel materials for specific applications can be expected to have a significant positive effect on the energy consumed in these processes. For example, catalytic materials are also extensively employed throughout the manufacturing industry to minimize toxic and environmentally undesirable emissions from automobiles, power plants, chemical plants and refineries. The development of more efficient catalytic materials and sensors for environmental applications will directly translate to benefits in human health and quality-of-life. Furthermore, the development of new sensor materials for specific biological compounds will result in the more efficient detection of human disorders and the development of improved pharmaceutical and food products, including but not limited to the development of improved cooking materials such as improved cooking oils. Another potential positive outcome from the improved discovery tools is sensors in the detection of toxins and explosives in our environments, and the related issue of our national security.

Combinatorial chemistry developments have revolutionized materials testing and evaluation procedures as well as the time required for the discovery of novel materials. Rather than screening each material sequentially, combinatorial methodology allows for the simultaneous testing of many new materials in parallel channel arrays. The typical combinatorial approach employed for the discovery of novel catalytic materials has been to measure the catalyst temperature and determine the catalyst efficiency in converting a targeted reactant to desired products (FIG. 1). This combinatorial approach allows for the screening of the maximum number of catalytic materials, which has been the primary objective of most combinatorial studies. In only a few cases have material characterization methodologies been applied to determine the catalytic materials' bulk and surface nature either before or after catalyst screening.

A primary objective of current combinatorial screening for new and novel materials is to enhance the discovery process. At present, this is mostly being achieved by screening each sample for the desired characteristic and, thus, as many samples as possible are now examined in a given period. However, this paradigm is rapidly reaching its asymptotic limit since hundreds of samples can already be robotically synthesized and analyzed on a daily basis.

For example, combinatorial methods in catalyst design have been primarily focused on improving catalytic efficiency. Additional combinatorial research in catalyst design has determined that bulk and surface structures as well as the properties of catalytic materials substantially affect reaction rates and are also dynamic variables that may equilibrate upon exposure to different environmental conditions. Current combinatorial strategies do not readily establish the molecular/electronic structure and activity/selectivity relationships that are essential to further accelerate the materials discovery process because information about the dynamic structures is not being collected. Current combinatorial chemistry approaches in the differing chemical areas, including but not limited to the area of catalytic materials discovery, have not incorporated the use of physical and chemical in situ and/or operando molecular and electronic spectroscopic methods or approaches to determine the dynamic bulk and surface nature of the catalytic materials as well as the presence and/or identification of any surface reaction intermediates during the screening process, do not establish the molecular/electronic structure, activity/selectivity relationships, and do not collect information on the dynamic structures and surface reaction intermediates, all of which can be the basis for more efficient materials discovery processes.

Other disciplines of science and engineering have developed methods of determining molecular information including optical spectroscopic methods such as Raman, IR, and UV-Vis. Recently, it has become possible to rapidly obtain such measurements in a matter of seconds due to significant instrumental advances. This opens up the opportunity to monitor molecular events during transient conditions such as pressure or temperature changes. Further, these optical spectroscopic methods also allow for surface mapping of materials due to their spatial resolution capabilities. The most spatially sensitive of these methods is Raman, which has spatial resolution capabilities to less than about a micron. IR has spatial resolution capabilities to about 10 microns. UV-Vis currently has spatial resolution capabilities to about 250 microns. Optical spectroscopic development has recently included development of capabilities to simultaneously obtain multiple measurements, but presently success has been limited in reports to combinations of two techniques.

### BRIEF DESCRIPTION OF THE DRAWINGS

The foregoing summary of the invention, as well as the following detailed description of illustrative embodiments, is better understood when read in conjunction with the accompanying drawings, which are included by way of example, and not by way of limitation with regard to the claimed invention.

FIG. 1 is a block diagram of a conventional combinatorial model.

FIG. 2 is a block diagram of an illustrative combinatorial model in accordance with at least one aspect of the present invention.

FIG. 3 is a perspective view of an illustrative combinatorial reactor system in accordance with at least one aspect of the present invention.

FIG. 4 is an illustrative representation of Raman shifts of selected sites which may be found on surfaces and in the bulk of catalytic materials.

FIG. 5 is a functional block diagram of an illustrative combinatorial material discovery system in accordance with at least one aspect of the present invention.

FIG. 6 is a perspective view of an illustrative reactor housing in accordance with at least one aspect of the present invention.

FIGS. 7, 8, and 9 are various alternative views of the reactor housing of FIG. 6.

FIG. 10 is a perspective view of an illustrative reactor channel in accordance with at least one aspect of the present invention.

FIG. 11 is a plane view of the reactor housing of FIG. 6 holding a plurality of reactor channels.

FIG. 12 is a plane view as in FIG. 11, and further showing an illustrative heating unit for heating the plurality of reactor channels.

FIG. 13 is a perspective view of another illustrative embodiment of a reactor assembly in accordance with at least one aspect of the present invention.

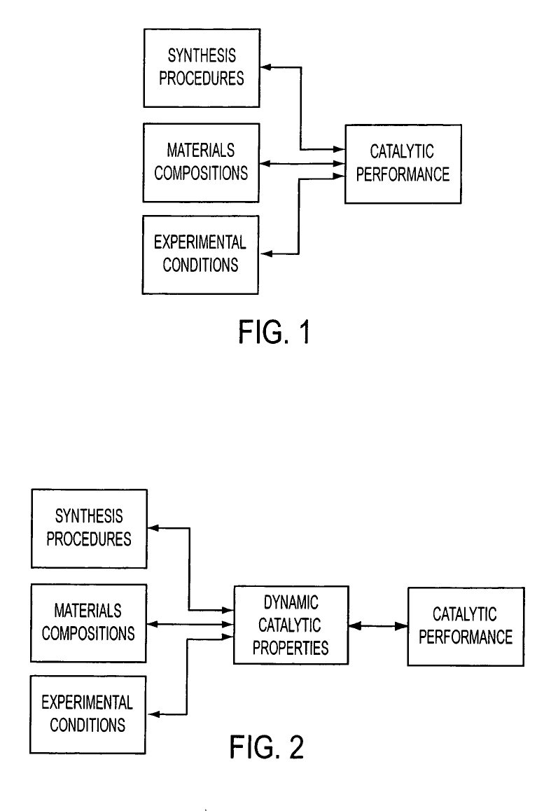

### SUMMARY OF THE INVENTION

In this discipline of chemistry, molecular and electronic structural and associated surface chemical kinetic and mechanistic information would be beneficial for acceleration of material discovery processes. Molecular-level information that may be useful in this acceleration includes, but is not limited to, the nature of the material (e.g., catalytic) active surface sites (molecular structure); surface reaction intermediates; surface complexes of reactants, intermediates, and products; bulk catalytic material structures; molecular and electronic structures and defects. Electronic information that may be useful in this acceleration includes, but is not limited to, the oxidation state of the cation, the cation's local and long-range coordination reflected by its band gap value, surface chemical kinetics and mechanisms and where applicable the affect on catalytic active sites due to bonding or coordination to an additional material with an independently varying band gap (e.g. nanomaterials). Further acceleration of the material discovery process can be accomplished by monitoring all stages, including but not limited to monitoring material composition synthesis procedures and experimental conditions. This monitoring in conjunction with combinatorial methodology will not only provide a large number of samples that can be rapidly screened, but also provide more relevant information on those samples thus leading to the design of improved materials with more beneficial properties in shorter amounts of time. Certain of the aspects of the present invention are beneficial to diverse fields of material research, including but not limited to catalytic research, biological research, and pharmaceutical research. Depending on the field of material research the operating parameters of certain aspects of the present invention will need to be controlled within the ranges whereby the materials and or reactions being studied are not adversely affected by the operating conditions. These parameters, including but not limited to temperature and pressure are well known to those of ordinary skill and the methods of their control or modification are design choices for those of ordinary skill that can be added on to certain aspects of the present invention.

Some aspects of the present invention are directed to a unique device and combinatorial method for targeted catalytic reaction screening of a plurality of catalytic materials simultaneously while determining the dynamic bulk and surface nature of the catalytic materials being screened, determining the molecular/electronic structure-activity/selectivity relationship of the catalytic materials or, collecting information on the dynamic structures of the catalytic materials. One of the aspects of the present invention is therefore a device and related methodology that accelerates the discovery process of novel materials and reduce the associated costs. This and other aspects of the present invention use analysis of dynamic molecular structure-activity relations along with combinatorial methodologies to guide the accelerated exploration of new and unique catalysts.

Some aspects of the present invention are directed to a unique device that in addition to the optical spectroscopies described herein also includes optical microscopy capabilities. Theses aspects of the present invention present a unique device that incorporates at least three optical spectroscopies and one optical microscopy material characterization techniques into a combination with a thermal or pressure transient spectroscopy characterization technique that measures system response to changes in temperature, pressure or partial pressure using systematic pulses or isotopically labeled molecules (e.g., Temperature Programmed Surface Reaction (TPSR) spectroscopy) into a single integrated device. These and other aspects of the present invention use the optical spectroscopic and microscopic characterization techniques to determine physical parameters of the material via the use of physical structural probes and use the thermal/pressure spectroscopy characterization technique to determine chemical parameters via the used of chemical probes that provide surface chemical kinetic and mechanistic information. Some additional aspects of the present invention use transient versions of thermal and pressure spectroscopy characterization techniques (e.g., TPSR) to provide more detailed information on the surface chemical kinetic and mechanistic processes, especially when evaluating steady-state catalytic reactions. Additional aspects of the present invention further enhance the information obtained by methods including, but not limited to, TPSR with the use of isotropic labels including but not limited to labels such as ²D, ¹⁸O, ¹⁵N, and ¹⁴C. Isotropic labels are currently used by those of ordinary skill in the art of catalytic studies to mark certain elements in order to determine location in product molecules, along with their affect on kinetics during the reaction.

Another aspect of the present invention provides a device and related combinatorial methods that allow a large number of catalytic materials to be screened simultaneously while using optical spectroscopic/microscopic methods in combination with chemical spectroscopy, such as TPSR, to provide information on the dynamic bulk and surface nature of the catalytic materials as well as, but not limited to information on surface species being screened under in situ or operando conditions. Another aspect of the present invention is to provide a device and related combinatorial methods that allow a large number of catalytic materials to be screened simultaneously while using Raman, IR, and UV-Vis spectroscopic along with optical microscopic methodologies in combination with chemical spectroscopy, such as TPSR, to assist in determining the dynamic bulk and surface nature of the catalytic materials being screened as well as, but not limited to information on the surface species, determining the molecular/electronic structure-activity/selectivity relationship of the catalytic materials, or collecting information on the dynamic structures of the catalytic materials and surface species under in situ or operando conditions.

In contrast, current combinatorial screening approaches have not addressed the simultaneous, or near-simultaneous, development of detailed molecular and electronic information on catalytic materials under reaction conditions. At least one reason for this is that implementation of such a complex protocol would have hampered the number of catalytic materials to be screened with conventional devices and using traditional combinatorial methods, to a point where the main driving force in combinatorial studies, maximum number of samples screened per unit time, is lowered below acceptable levels. Combinatorial characterization methodologies have therefore been primarily developed to examine catalysts only before and after catalytic reactions. However, the catalytic active materials and its associated surface species under reaction conditions are generally different than the catalytic materials and associated surface species present before or after catalytic reactions, thus leading to information that has limited value in materials development. Some aspects of the present invention use operando spectroscopy to evaluate, analyze or measure the properties of the catalytic material and its associated surface species during the reaction thus providing a greater quantity of detailed information that further accelerates material research. Additionally, current combinatorial methods have failed to integrate chemical spectroscopy (e.g., TPSR) due to focusing on maximum number of samples processed and current interest being limited to steady-state performance.

Chemical spectroscopy methods, including but not limited to TPSR, further enhance the chemical information available from steady-state studies by providing information useful in the development of activity and/or selectivity relationships. TPSR generally is used to determine the temperature response of reactions in order to provide information usefully to deduce catalytic reaction mechanisms and kinetics. TPSR spectroscopy devices generally consist of a chemical probe molecule with a defined temperature rise as a function of time profile in which reaction products are detected as a function of temperature by mass spectrometry. TPSR spectroscopy can be used to provide information useful in deduction of reaction mechanisms, bonding mechanisms between adsorbates and the adsorbing surface and functional group nature. TPSR spectroscopy can be used to measure properties under reaction conditions and is generally applied in two ways when studying kinetics of active catalytic surface sites, including but not limited to rate determining step determinations, reaction order and activation energy. One manner of TPSR spectroscopy is to coadsorb gases on a catalyst surface after which heating is done with an inert carrier gas (e.g., He). Another manner uses a catalyst with preadsorbed surface species which is subsequently heated in a reactive carrier gas (e.g., CO). This manner of TPSR can also provide information useful for quantitative determination of adsorbate coverage. Some aspects of the present invention combine TPSR spectroscopy with the optical spectroscopy and microscopy described above to determine dynamic bulk and surface nature of the catalytic materials being screened as well as, but not limited to information on the surface species, determining the structure-activity/selectivity relationship of the catalytic materials, or collecting information on the dynamic structures of the catalytic materials and surface species under in situ or operando reaction conditions. Similarly, other chemical spectroscopic techniques may be used to create pressure or partial pressure transients and measure a systems reaction thereto in a similar fashion as TPSR.

Another aspect of the present invention provides for one or more combinatorial catalyst development libraries (from the chemical and optical spectroscopies described above) that may be developed using the devices and methodologies according to other aspects of the invention. A searchable library of the molecular-based information process can decrease the number of future samples to be screened and improve economic efficiency along with accelerating timelines for future discovery processes. Generally, the library would store the molecular-based information that provides a fundamental basis for understanding the targeted reaction. Further, the fundamental molecular structural information may allow the use of the molecular/electronic structural-physical and chemical relationships in other targeted applications. Over time, it is expected that the use of such molecular/electronic structure-property libraries for other targeted applications may further decrease the number of samples that will need to be screened and further accelerate the discovery of novel materials.

These and other aspects of the invention will be apparent upon consideration of the following detailed description of illustrative embodiments.

### DETAILED DESCRIPTION OF ILLUSTRATIVE EMBODIMENTS

It is noted that various connections are set forth between elements in the following description. It is noted that these connections in general and, unless specified otherwise, may be direct or indirect and that this specification is not intended to be limiting in this respect. It is further noted that as used herein, the term in situ refers to characterization of a catalytic material under any controlled environment (e.g., temperature, vacuum, pressure, oxidation, reduction or reaction) and the term operando refers to the simultaneous characterization and activity/selectivity measurements of a catalytic material under relevant (e.g., industrial) reaction conditions.

One aspect of the present invention is to collect and analyze information on a material's dynamic bulk and surface characteristics, and surface species, along with its catalytic performance properties under reaction conditions. Exemplary aspects of the present invention as applied to catalytic reactions are described in detail. As known to those of ordinary skill catalytic systems generally include gas-solid, liquid-solid, or gas-liquid-solid phase systems and also include complex catalysts such as a soluble homogeneous catalyst, enzyme or protein. The exemplary aspects of the present invention with gas-solid systems do not limit aspects of the current invention to other catalytic systems. Previous combinatorial approaches (as shown in Fig. I) focused on the number of materials to be rapidly screened in the experimental space. Another aspect of the present invention is to maintain a combinatorial methodology to rapidly screen materials but also to combine that methodology with unique aspects of optical spectroscopy to obtain detailed molecular and electronic structure or property information under reaction conditions. In other aspects of the invention, the collection and storage of this information in searchable databases may lead to the molecular engineering of advanced catalytic materials in combinatorial studies, as well as in conventional catalysis research including the use of aspects of the current invention to design catalytic active surface sites for specific reactants and significantly decrease the number of samples that will need to be screened for future catalytic developments. Yet another aspect of this invention relates to novel physical and chemical molecular/electronic spectroscopic tools to enhance the discovery of catalytic materials during combinatorial chemical screening.

In order to address the numerous shortcomings of current combinatorial chemical screenings, a novel combinatorial system according to various aspects of the present invention simultaneously provides in situ and/or operando physical spectroscopic measurements of catalytic materials under relevant (e.g., industrial) reaction conditions. The specific optical spectroscopic characterization methods provide: 1) molecular structural information under high temperature (T) and high pressure (P), 2) electronic structural information under high T and high P, 3) real-time analysis for temporal resolution, and/or 4) spatial resolution for surface mapping. The optical spectroscopic characterization methods may include, but are not limited to Raman, IR, and UV-Vis. and their respective Fourier transform (FT) equivalents. These may also be used in combination with either optical microscopy, chemical spectroscopy (e.g., TPSR), or both.

The molecular information provided by aspects of the current invention generally includes the nature of the molecular structure of the catalyst. For example, the molecular and electronic structural information provided by aspects of the invention may include the nature of the catalytic active surface sites, the nature of the surface species (e.g., reaction intermediates), and the bulk catalytic materials (e.g., structures).

The electronic information provided by one or more aspects of the invention generally refers to the number and distribution of electrons for various atoms on the catalyst surface. The electronic information may include, but is not limited to, one or more of the following: (1) the oxidation state of the cation; (2) the cation's local coordination (e.g., the number of M—O, M—O—M and M—M bonds); (3) the cation's long-range domain size (e.g., monomer, polymer, cluster coordination); and (4) the electronic structure of the substrate to which the cation or complex is bound.

The operando approach of one aspect of the invention illustratively shown in FIG. 2 may quickly and accurately provide the most fundamental information about a particular catalytic material for a targeted reaction, including but not limited to the surface kinetics of the reaction under investigation, the nature of the surface intermediates, the selectivity at different reaction conditions, and information on the bulk and/or surface molecular and electronic structures of the catalyst that give rise to the observed activity and the selectivity. This information, in combination with transient investigations of the targeted reaction, provide a basis for developing additional surface kinetic information as well as mechanistic insights such as heats of adsorption and equilibrium rate constants of adsorption. The transient investigations may use chemical spectroscopy techniques (e.g., TPSR spectroscopy).

The individual optical and chemical spectroscopic characterization methods discussed herein are generally available to those of ordinary skill in the art using commercial embodiments of those methods that are, on their own, publicly available for purchase. Dual optical spectrophotometer systems are also publicly available and are generally either based on FT-IR or dispersive Raman platforms that are modified by manufacturers to provide the other spectroscopic system. While either basic platform may used to accomplish certain aspects of the invention, some aspects of the invention are better served using the dispersive Raman platform due to issues associated with temperature restrictions and data loss from amorphous and surface phases in the FT process. The selection of specific spectroscopic platform manufacturers is simply a design choice based on familiarity of operation for those of ordinary skill in the art. For example, in some of the aspects of the invention the Raman and IR spectroscopic instruments use a combined Raman and IR spectroscopic instrument with a confocal microscope for spatial resolution. An example of such an instrument is currently publicly available on the Internet at wwwjobinyvon.com, which has a confocal microscope that provides a total of three optical spectroscopic and microscopic techniques. As is well known to those of ordinary skill, the simultaneous Raman and IR measurements can be achieved by alternating the Raman and IR apertures every second by a small shutter. Other methods of alternating the Raman and IR apertures are known in other publicly available devices.

Another aspect of the present invention is to provide a single device with the capabilities to address all optical spectroscopic requirements as well as surface chemical kinetic and mechanistic capabilities through use of the combination of various methods of optical spectroscopy, optical microscopy and chemical (e.g., TPSR) spectroscopy; or at least a larger combination of spectroscopic requirements than is provided in the prior art. In some aspects of the invention, this is generally accomplished by modification of a combined Raman/IR system to also have the capability to measure the optical UV-Vis signal in a combinatorial screening system. In some aspects of the invention, this modification may be accomplished by modifying the confocal microscope of the Raman/IR device to allow the introduction of UV-Vis fiber optic sensors. Uv-Vis fiber optic sensors are well known to those of ordinary skill and exemplary devices are publicly available on the Internet at www.avantes.com.

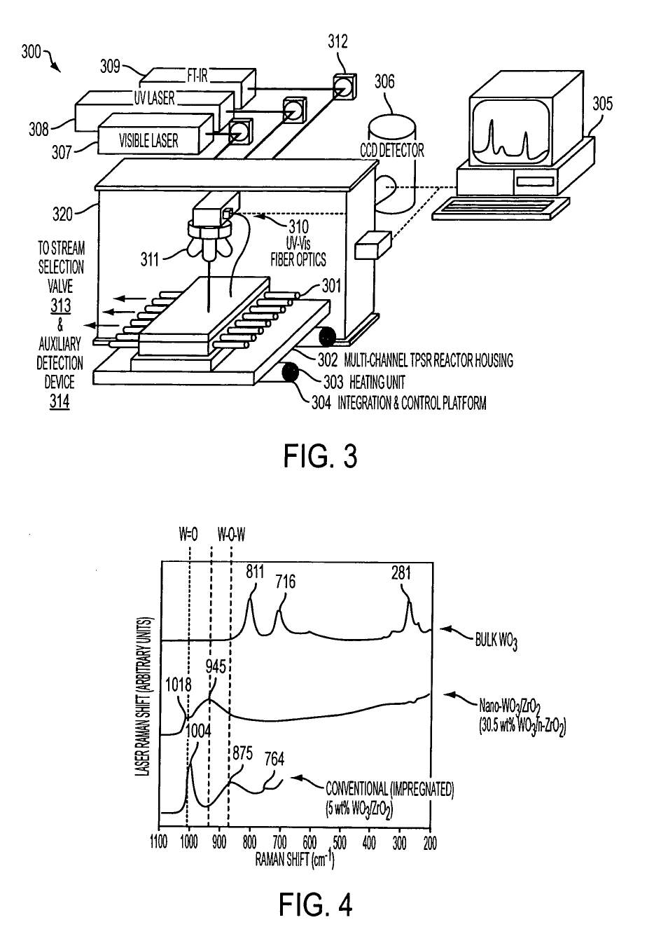

Generally, the Raman/IR device is modified so that a UV-Vis fiber optic system is simultaneously functional with the existing Raman/IR devices yet does not interfere with the corresponding Raman and IR measurements. An overview of such a combinatorial reactor system 300 is illustratively described with reference to FIG. 3. The UV-Vis optical fiber is generally integrated with the Raman/IR confocal microscope. For example, the optical fiber may be inserted into the white light reflection illumination port of the confocal microscope. As is well-known, the US-Vis optical fiber probe generally is provided with its own light source, signal collector and spectral analyzer, and its further integration with the Raman/IR software can also be easily readily achieved if so desired. For instance, such probes exist that include a central optical fiber around which are hexagonally arranged a plurality of smaller optical fibers. The smaller optical fibers provide the excitation UV-Vis light while the larger optical fiber collects the scattered and/or reflected UV-Vis light from the target for later analysis. Alternatively, the probe may be mounted outside the confocal microscope.

Regardless of the physical arrangement of the UV-Vis probe, collection of information using the UV-Vis probe generally should be accomplished while the Raman laser is in the off position (or while the light from the Raman laser is attenuated or blocked) to avoid optical interference. Similar optical interference may occur and should similarly be avoided from the Raman laser when attempting IR measurements. Preferably, this may be accomplished by, e.g., a multi-aperture shutter system. Such aperture systems are known, but only currently available for use with Raman and IR measurements being made by the same system. Accordingly, the multi-aperture shutter system used herein may be modified or originally constructed to control at least three separate apertures to open and close in a synchronized manner. For instance, each of the three apertures may be configured to be sequentially opened (while each of the other apertures is closed) over a short period of time. The period of time may be any desired, such as but not limited to a few seconds. For example, over a short timeframe, the system may cycle, one at a time, between Raman, IR, and then UV-Vis measurements during steady-state catalytic studies.

Transient TPSR spectroscopy studies are generally performed after adsorption of the reactants on the catalyst at mild temperatures (e.g., about 100 degrees Celsius or less), followed by flushing out with an inert gas (e.g., N₂, He, Ar, etc.) of any residual gas-phase molecules. The flushing-out is generally followed by incrementally increasing the reactor temperature at a constant rate (e.g., by 1-10 degrees Celsius/minute) and in-flowing one or more gases including but not limited to reactants, products, He, or He/O₂ mixture. The gas flow rate affects the efficiency of flushing-out materials such as desorbed reaction products and unreacted products for later spectroscopic analysis, preferably by mass spectroscopy.

As seen in the illustrative diagram of FIG. 3, combinatorial reactor system 300 may allow simultaneous spectroscopic screening of multiple catalytic materials for a specific reaction. Combinatorial reactor system 300 may further allow the feed gas composition and flow rates to each of a plurality of reactor channels 301 to be independently varied. One or more excitation sources, along with various supporting optics, may provide incident radiation onto the samples in reactor channels 301. For example, combinatorial reactor system 300 may include a visible laser source 307, a UV laser source 308, an IR source 309, and/or a UV-Vis excitation source for optical fibers 310, wherein the excitation radiation of each may be directed into reactor channels 301. Optics for directing or otherwise guiding the excitation radiation may include various mirrors, filters, and/or optical guides, such as one or more lenses 311, one or more mirrors such as mirror 312, and/or one or more UV-Vis optical fibers 310. Combinatorial reactor system 300 may further include real-time online analytical instrumentation for spectral analysis, which may be embodied as a computer 305.

Computer 305 may perform such analyses as mass spectrometry data, IR data, Raman data, or gas chromatography (GC) data, to simultaneously monitor the exiting gases to determine the steady-state and/or transient catalytic activity and selectivity from each of reactor channels 301. The data derived from monitoring the exiting gases may be sensed by a detection device 314 that may be part of or physically separate from system 300. In addition, the exiting gases may also be switched between a vent and detection device 314 using a stream selection valve 313. Computer 305 may further be provided with output from one or more optical detectors, such as a charge-coupled device (CCD) detector 306 or detectors that are part of devices 307, 308, and 309.

Reactor channels 301 may be partially or fully disposed within a reactor housing 302. Reactor housing 302 is physically coupled to (e.g., mounted on) an integration and control platform 302. Reactor housing 302 may be arranged horizontally or vertically, or at any other angle, and its physical placement may be dynamically controlled through motorized control of platform 304. Platform 304 may move reactor housing 302 in any of X, Y, and/or Z directions so as to place reactor housing 302 in proper relation to the various optics that transport the excitation radiation. Alternatively or additionally, the optics may be dynamically adjusted to provide proper relation to the physical placement of reactor housing 302 and to provide for axial measurement along reaction tubes further described below. A heating unit 303 or other heating unit may be disposed proximate to reactor housing 302 so as to heat the substances within reactor channels 301.

In operation, detection device 314 and/or one or more of the optical detectors 306, 307, 308, and 309 may take measurements of the catalytic reactions occurring in each of reactor channels 301 (and/or in reactor chambers that are part of rector channels 301, as will be discussed further below). In particular, detection device 314 may take physical measurements of the gases that exit through reactor channels 301, and optical detectors 306, 307, 308, and 309 may take optical measurements of the actual surfaces of the catalysts involved in the catalytic reactions and disposed in the reactor chambers. Optical parameters are also measured along the axial and radial directions of the reactor tube as any change in gas phase composition may affect the molecular and electronic structures and surface sites. The UV laser 308 excitation may also simultaneously yield Raman vibrations of gas phase molecules, such as doubly-bonded O₂, triply-bonded N₂, etc. The various measurements by these various optical and non-optical detectors may occur in rapid succession in relation to one another to avoid optical crosstalk of their signals, and in any event may all occur during the same catalytic reactions. These measurements may occur as a single measurement sample, or as a series of measurement samples over time, during the progression of the catalytic reactions even though the catalytic reactions are progressing in a continuous manner and are not quenched prior to or during the measurements.

In order to more fully establish the molecular and electronic structure-activity/selectivity relationships for the catalytic materials, it may also be preferable to obtain complementary chemical characterization information about the active surface sites of catalytic materials. Generally, this information may be obtained using chemical probe molecules including, but not limited to, methanol. While some aspects of the present invention use methanol, some aspects of the invention for different catalyst investigations at different operating characteristics may use different chemical probe molecules. Molecular and electronic structural (e.g., oxidation state) libraries for the physical characterization methods may also accelerate the Raman, IR and UV-Vis assignments of materials as well as the chemical spectroscopy (e.g., TPSR) libraries may possess the complementary chemical information to assist in the identification of the molecular and electronic structures of the catalytic active surface sites and their cation oxidation states.

In one aspect of the invention, CH₃OH was used to provide important information about the nature of catalytic surface sites including, but not limited to, the number of active surface sites, the types of surface sites (redox, acidic or basic) and the number of molecules converted per active surface site per second (a.k.a, TOF values) for each type of surface site. The number of active surface sites can be determined by any number of methods known to those of skill in the art. In an illustrative embodiment, methanol chemisorption at temperatures where physically adsorbed methanol is not present on the surface, and only dissociatively chemisorbed methanol is present as surface methoxy species (typically 100° C.), may be used. Using the method of this aspect of the invention, the methanol reaction products reflect the different types of surface sites: HCHO from surface redox sites, CH₃OCH₃ from surface acidic sites, and CO/CO₂ from surface basic sites. The TOF values for the different reactions paths are obtained by dividing each of the reaction rates for product formation by the number of active surface sites. Thus, the CH₃OH chemical probe studies provide rich chemical information about the nature of catalytic active surface sites on a catalyst surface.

It is well known to those of ordinary skill that CH₃OH-Temperature Programmed Surface Reaction (TPSR) spectroscopy may provide chemical information about the identity of the active surface sites, their oxidation states on catalytic surfaces and participation of bulk lattice oxygen in catalytic reactions. It is for these reasons that in some aspects of the present invention a TPSR combinatorial system may be desirable to also provide insights about the oxidation states of surface catalyst cations. As illustrated below, various aspects of the invention may use TPSR spectroscopy to preliminarily determine the oxidation states of vanadia cations. Other preliminary studies for several bulk and niobia supported oxides, where the active component is deposited on a niobia substrate, have successfully demonstrated that the CH₃0H-TPSR specific product and peak temperature, Tp, reflect the specific surface cation present on the catalytic material surface and the preliminary data are shown below:

| CATALYTIC MATERIAL | Tp (° C.) | REACTION PRODUCTS |
|---|---|---|
| V₂O₅ (V⁺⁵) | 185 | HCHO |
| Supported V₂O₅/Nb₂O₅ (V⁺⁵) | 185 | HCHO |
| Supported V₂O₅/Nb₂O₅ (V⁺⁴) | 201 | HCHO |
| MoO₃ (Mo⁺⁶) | 195 | HCHO |
| MoO₃ (Mo⁺⁵) | 212 | HCHO |
| MoO₃ (Mo⁺⁴) | 225 | HCHO |
| Supported MoO₃/Nb₂O₅ (Mo⁺⁶) | 192 | HCHO |
| Supported MoO₃/Nb₂O₅ (Mo⁺⁵) | 212 | HCHO |
| TeO₂ | 432 | HCHO/CO₂ |
| Supported TeO₂/Nb₂O₅ | 260 | HCHO/CO₂ |
| Nb₂O₅ | 300 | CH₃OCH₃ |

The above data reveal that the Tp temperature and product formation reflect the nature of the active surface sites (the specific element) and oxidation states. The reduced sites were formed by stoichiometric reaction of the surfaces with methanol. The surface V and Mo sites behave as surface redox sites, the surface Nb sites behave as surface acidic sites and the surface Te sites behave as surface redox-basic sites. The relative reactivity of these surface cations is V>Mo>>Nb>Te. Furthermore, the surface Te sites are dramatically promoted by their coordination to the niobia support (Tp decreases by ˜170° C.). Interestingly, the absence of dimethyl ether production from surface acidic sites for the metal oxides deposited on Nb₂O₅ support reveal that there were no exposed or a small number surface Nb cations present in the synthesized materials. Application of the novel approach encompassing aspects of the present invention to the bulk mixed Mo—V—Nb—Te—O metal oxide system, showed that the optimum catalytic material should have both surface redox (V⁺⁵, Mo⁺⁶) and acidic sites for propane oxidation to acrylic acid. CH₃OH-TPSR experiments in the absence of gas phase O₂ also revealed that the oxygen directly involved in oxidation reactions of this catalyst originates from the bulk lattice of the mixed metal oxide, probably by a Mars-van Krevelen mechanism. Comparative studies with bulk V₂O₅ and MoO₃ also showed that bulk lattice oxygen is much more mobile in V₂O₅ than in MoO₃ because surface V⁺⁵ was always present due to reoxidation by the bulk lattice oxygen and surface Mo was not reoxidized by the lattice oxygen.

CH₃OH-TPSR libraries for the surface reactivity and oxidation states do possess an inherent technical risk. Although the preliminary studies demonstrate that the surface Mo, V, Te and Nb cations and their oxidation states can be discriminated by CH₃OH-TPSR, it is not yet clear if there is significant overlap in Tp and similar reaction products among a larger set of cations. Such a scenario would compromise the ability of CH₃OH-TPSR to identify surface elemental and oxidation states. To minimize such complications, the CH₃OH-TPSR experimental conditions may need to be modified and perhaps others will have to be examined for their potential to chemically discriminate among the various cations and their oxidation states. The success of overcoming this technical hurdle is likely very good because of the wide temperature range of the reactions and the specific reaction products formed from the different surface cations. The successful development of the CH₃OH-TPSR surface characterization system will provide an inexpensive method and non-vacuum technique to determine the elemental surface composition and surface cation oxidation states of materials. This information may be important for those materials where the interfacial properties control their performance. Aspects of the present invention may thus greatly accelerate combinatorial, materials research and materials evaluation studies that focus on interfacial properties.

TPSR spectra may also possess quantitative kinetic information about the rate determining step of a catalytic reaction, which is contained in the Tp value. The combination of this surface kinetic rate constant with corresponding steady-state catalytic studies allows for the direct determination of the adsorption equilibrium constant and the thermodynamic surface heat of adsorption. Further, the order of appearance of reaction products and intermediates during such a transient experiment directly reveals the mechanistic elementary surface steps taking place during the surface reactions. The surface kinetic, thermodynamic and reaction mechanism information can be used to develop molecular-based models of the catalytic events for a targeted reaction. TPSR catalytic experiments may be performed with any targeted molecule(s) to better determine the molecular events and surface requirements as long as the reactant(s) can be adsorbed on the surface of the catalytic material at modest temperatures. There may be situations where one of the reactants cannot be easily adsorbed on the catalyst surface. For example, where weakly adsorbing propane is used during propane ammoxidation. In this example, the second reactant (NH₃) may be adsorbed on the catalyst surface and the propane kept in the gas phase during the TPSR experiment. Isotopic tagging of specific functionalities may further be used to enhance the mechanistic details obtained from various aspects of the present invention.

The methanol probe reaction may be employed for either steady-state or pulsed mode so as to periodically monitor the state, including the state of the catalyst life or the state of the catalyst after a regeneration procedure, of the catalytic material surface as a function reaction time for a specific reaction. This may allow for the rapid online monitoring of the changes at the surface of catalytic materials due to sintering, poisoning, coking, surface composition or surface cation oxidation states. Small methanol pulses may also be introduced during many catalytic reactions to determine the state of the surface of the catalytic materials during different reaction environments.

An illustrative non-combinatorial methodology is now described showing how the innovative device and methodology may be used to discover a new class of catalytic materials. Specifically in this example, new nano-catalytic materials are identified. As is well known in the art, supported WO₃/ZrO₂ catalysts possess significant surface acidity, and there is much interest in developing these catalytic materials as solid acids for isomerization reactions of petroleum fractions to increase their octane values (e.g., n-pentane to isopentane). However, the surface acidity of the surface WOx sites in conventional nano-supported WO₃/ZrO₂ catalyst are not active enough to conduct this reaction under industrial conditions, and carbon deposition further aggravates the catalytic activity because of significant catalyst deactivation. With reference to FIG. 4, Raman analysis of the conventional catalyst reveals that the surface WOx species are present as isolated and polymerized surface species on the ZrO₂ support. When a nano-supported WO₃/ZrO₂ catalyst is synthesized on 5 nm ZrO₂ particles by employing aqueous ammonium metatungstate and a nonionic triblock copolymer surfactant (called P123) templating agent, the Raman spectrum reveals a very different surface WOx molecular structure. The surface WOx species on the nano-ZrO₂ support primarily possess a new polymerized surface WOx structure that doesn't possess many terminal W═O bonds (the remaining small terminal W═O bonds at ˜1000 cm⁻¹ are believed to originate from residual isolated surface WOx species). In situ Raman and UV-Vis measurements in alkane environments reveal that, unlike the conventional supported WO₃/ZrO₂ catalyst that is only mildly reduced and covered with carbonaceous deposits, the surface WOx species on the nano-ZrO₂ support are almost completely reduced to a lower oxide (primarily W⁺⁵) and are free of carbon. The different responses of the conventional and supported WO₃/ZrO₂ catalytic materials reveal that the different surface WOx structures possess different chemical properties.

In this example, the surface reactivity of this interesting surface WOx species on nano-ZrO₂ is further chemically probed with CH₃OH-TPSR to determine their behavior in acidic reactions. The Tp temperature for dimethyl ether formation, the acidic product (100% selectivity), is found to dramatically decrease by ˜50° C. indicating about a 30 fold increase in the rate constant for this acidic reaction compared to the conventional supported WO₃/ZrO₂ catalysts. In light of the above findings of a new surface WOx molecular structure, its enhanced surface reactivity and lack of carbon deposition, this novel material is examined for n-pentane isomerization to iso-pentane. The steady-state n-pentane catalytic studies reveal that the novel nano-supported WO₃/ZrO₂ catalysts are greater than 50 times more active per gram of catalyst (>10 times more active per m²) and 100% selective for n-pentane isomerization than the conventional catalyst. This example also illustrates that development of substantially identical information can be accomplished at an accelerated rate using the combinatorial apparatus and methodology described herein.

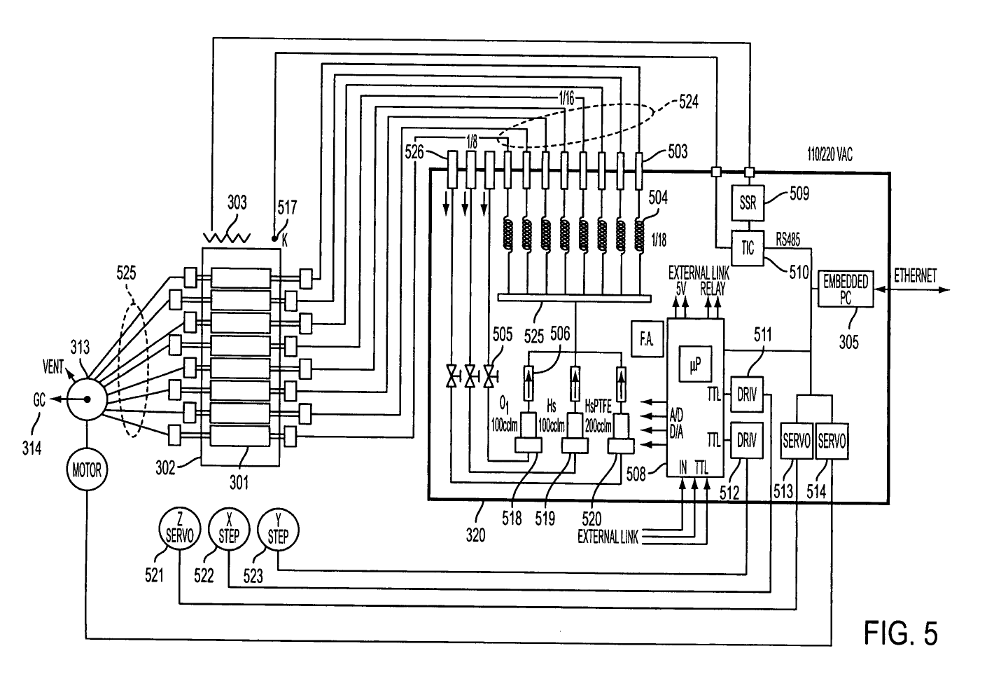

Referring to FIG. 5, an illustrative functional block diagram of such a combinatorial apparatus, called herein a combinatorial reactor system 300, is shown. Reactor channels 301 are each provided individual gas supplies via a parallel set of supply tubing 524 and are each provided with drains via a set of drain tubing 525. On the supply side, one or more sources may be provided that supply the various gases used in the chemical reactions in reactor channels 301. For example, gaseous oxygen and helium may be provided via input ports such as port 526 and control valves such as valve 505. Each source may have a respective regulator 518, 519, 520, as well as a respective flow meter that indicates the amount of flow, such as flow meter 506. The gases are mixed at a mixer 504, and supply tubing 524 then exits housing 320 via ports such as port 503. Capillary tubes 504 are also provided to equalize distribution of incoming gases to reactor channels 301. On the drain side, drain tubing 525 is coupled to either a vent or to detection device 314, which in this example is a gas chromatograph, in accordance with the position of stream selection valve 313. Stream selection valve 313 is selectable between positions by a servo motor 501. Servo motor 501 is controlled by a servo controller 514, which in turn is controlled by computer 305.

As previously mentioned, reactor channels 301 is heated by heating unit 303, which may provide a variable amount of heat as desired. A sensor 517 detects the current temperature of heating unit 303 and/or an area near heating unit 303. Sensor 517 provides a signal to a temperature indication and control (TIC) unit 510. Based on the feedback signal, TIC 510 controls a solid-state relay (SSR) 509 to switch between on and off states, which in turn regulates whether heating unit 303 generates heat. In this way, the average temperature may be accurately controlled. TIC 510 may also be controlled by and/or provide temperature information to computer 305 via an RS-485 serial connection.

Computer 305 and/or a processor 508 may be used to control some or all of the functions of combinatorial reactor system 300. Computer 305 and/or processor 508 may each include a microprocessor, as well as one or more transistor-transistor logic (TTL) ports. The microprocessor may operate at a relatively high speed. For example, modern microprocessors presently operate with a clock speed in the multi-GHz range. The TTL ports may drive one or more external devices, such as motors. For example, processor 508 may control drivers 511 and 512, which in turn control an X stepper motor 522 and a Y stepper motor 523. A Z servo motor may also be controlled by processor 508 via a servo controller 513. Together, the three motors 521, 522, 523 control the position of platform 304 along at least three translational degrees of freedom X, Y, Z. In addition, platform 304 may be rotated about one or more rotational axes. As previously mentioned, reactor housing 302 moves with platform 304. Processor 508 and/or computer 305 may be used to synchronize and control the multi-aperture shutter system as previously described.

Any of the elements discussed in connection with FIG. 5 may be fully or partially enclosed within housing 320, or may be outside of housing 320. For example, although computer 305 is shown in FIG. 3 as being external to housing 320, computer 305 may be disposed fully or partially within housing 320 as shown in FIG. 5.

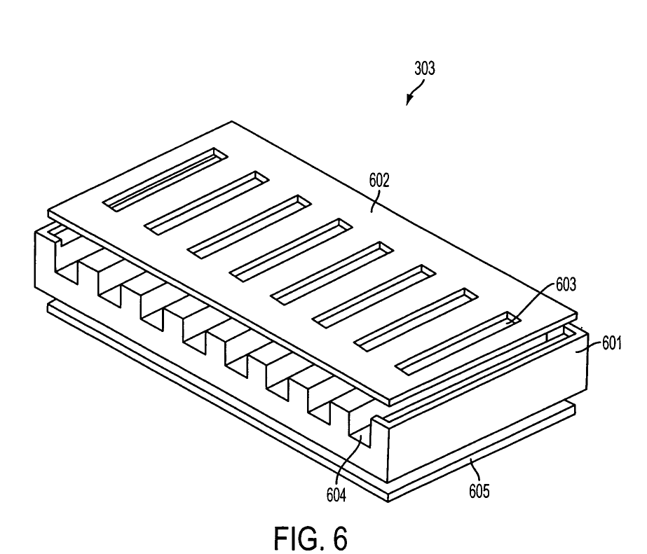

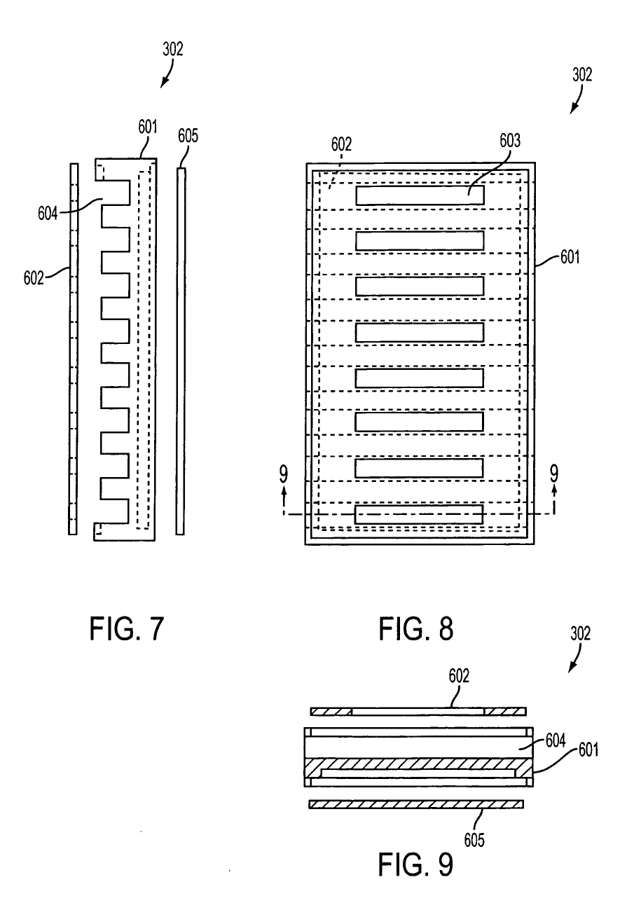

Referring to FIGS. 6, 7, 8, and 9, various views of an illustrative embodiment of reactor housing 302 are presented. Reactor housing 302 includes a base portion 601 and an upper plate 602 configured to fit against base portion 601. Base portion 601 is generally in the form of a block having a plurality of parallel elongated grooves 604 in which reactor channels 301 may be placed. For example, base portion 601 may have outer dimensions of approximately 122 millimeters in length by about 18.2 millimeters in depth by about 65 millimeters in width. A bottom plate 605 may also be coupled to the side of base portion 601 opposing upper plate 602. Bottom plate 605 may have dimensions of, for example, about 118 millimeters by about 61 millimeters by about 2.5 millimeters in thickness.

In the shown embodiment, base portion 601 has eight parallel grooves 604. However, any number or shape (e.g., rectangular, cylindrical, triangular, or other geometric shape) of grooves may be formed, depending upon the number and shape of reactor channels 301 needed. Grooves 604 may have dimensions of, for example, about 7.5 millimeters in width by about 7.5 millimeters in depth, and extend fully across to opposing sides of base portion 601. In addition, grooves 604 may extend in parallel with each other with a spacing of about, e.g., 14.4 millimeters between the axial centers of neighboring grooves 304 (i.e., in this embodiment, about 6.9 millimeters between neighboring groove edges). When base portion 601 and upper plate 602 are positioned so as to fit together, upper plate 602 at least partially covers one side of grooves 604 to form elongated channels bounded by base portion 601 and upper plate 602 and open at opposing ends of reactor housing 302. Because upper plate 602 is removable and connectable with base portion 601, reactor channels 301 may easily be moved and inserted into grooves 604.

Upper plate 602 is in the form of a substantially flat, thin, and planar member, and may have the same dimensions as bottom plate 605. Upper plate 602 has a plurality of slots 603 formed fully through upper plate 602. Slots 603 may be elongated, and may have dimensions of, for example, about 37 millimeters in length by about 5 millimeters in width. When base portion 601 and upper plate 602 are positioned so as to fit together, each of slots 603 is longitudinally aligned with a different respective one of the grooves 604. Thus, slots 603 effectively form windows aligned with grooves 604 that allow excitation radiation to be incident on reactor channels 301 when positioned within grooves 604.

Some or all of reactor housing 302 may be constructed from a partially or fully transparent material, such as diamond or quartz, to provide for at least partial optical transparency, thereby allowing excitation radiation to be incident on the materials involved in the chemical reaction of interest as well as the sensors to be able to measure the reaction. Alternatively, reactor housing 302 may be constructed from an opaque material such as metal (e.g., zinc selenide). The particular material(s) that reactor housing 302 is constructed from preferably should be balanced, however, with stringent temperature and pressure requirements for reactor channels 301 during steady-state and transient temperature (from ambient to 1000° C.) and pressure studies. For example, a moving slit may be utilized for optical analysis of various points within the chemical reaction area to limit heat loss and maintain the materials at the desired temperature.

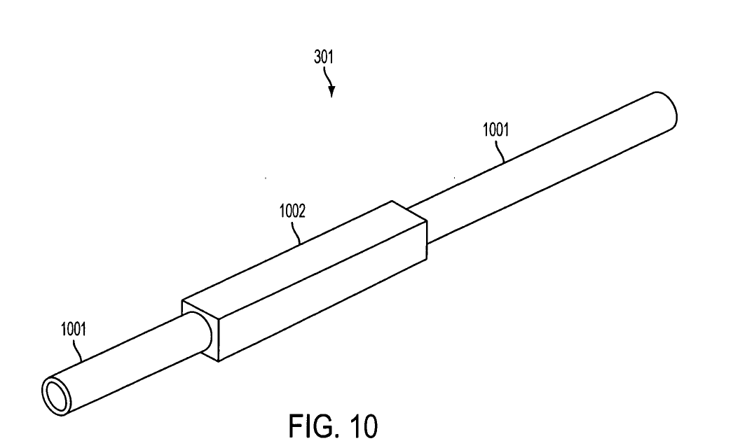

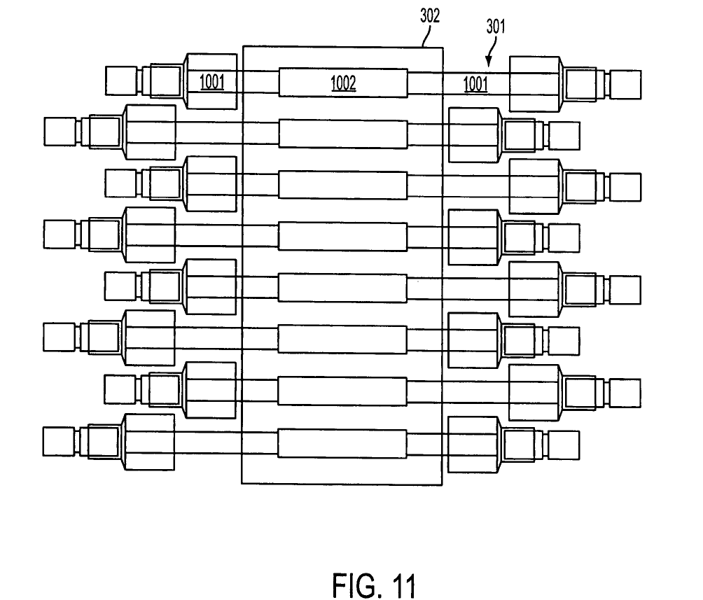

Referring to FIGS. 10 and 11, each reactor channel 301 is elongated and may have a reaction chamber 1002 with end portions 1001 longitudinally arranged on opposing sides of reaction chamber 1002. Reaction chamber 1002 may have dimensions of, for example, about 7.5 millimeters in width (the same as, or slightly less than, the width of grooves 604) and about 42 millimeters in length. Reaction chamber 1002 may have a generally rectangular or other outer shape that cooperatively mates with the inner shape of grooves 604. Reaction chamber 1002 may be where chemical reactions of interest take place. Thus, when cooperatively mated with one of grooves 604, reaction chamber 1002 will be aligned so as to be visible through one of slots 603. The purpose of reaction chamber 1002 is to hold the catalyst during the catalytic reaction. Accordingly, it may be desirable that reaction chamber 1002 be at least partially, if not fully, optically transparent, so that optical measurement devices 306, 307, 308, and 309 may obtain an optical view of the catalyst disposed within reaction chamber 1002.

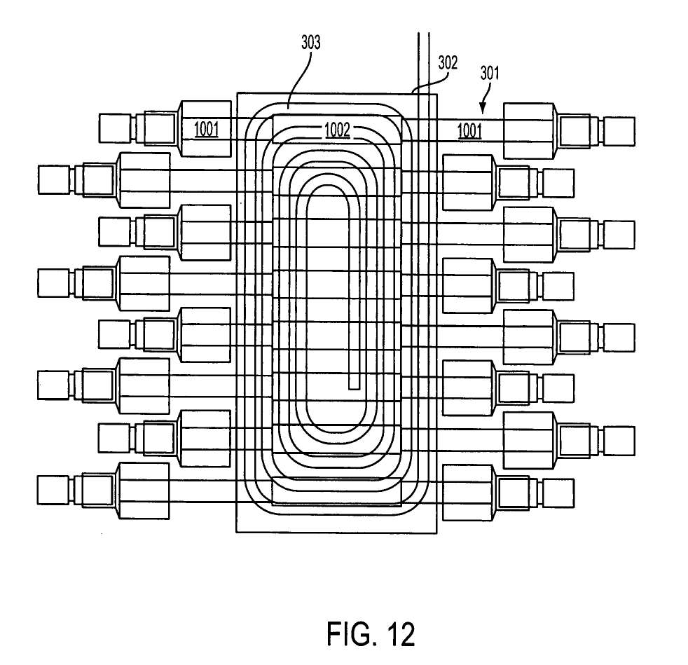

Referring to FIG. 12, reactor housing 302 and reactor channels 301 are shown in conjunction with heating unit 303. The point of view of FIG. 12 being from the top looking down, heating unit 303 is disposed underneath reactor housing 302. Heat from heating unit 303 travels up through reactor housing 302 and into reactor channels 301. Heating unit 303 is shown as a resistive-type heating element, however any type of heat source may be used.

Thus far has been described an embodiment where platform 304, reactor housing 302, and reactor channels 301 are arranged horizontally. However, platform 304 may be configured in a vertical arrangement rather than the horizontal arrangement shown in FIG. 3. A vertical arrangement may be used to help avoid gas bypassing in the fixed-bed reactors and may also help to reduce heat transfer to spectrometer microscope lens 311, which can be damaged by extreme temperatures. Any heat flux between reactor channels 301 and microscope lens 311 may be controlled by cooling reactor channels 301 with a circulating fluid. A cooling mechanism may be desired at higher reaction temperatures, such as those exceeding 450 degrees Celsius. Such cooling mechanisms are well-known to those of ordinary skill in the art. For example, a commercial cooling cell is presently available at http://www.linkham.com. In addition, reactor housing 302, platform 304, and the various optics may be configured as appropriate to operate in such a vertical arrangement.

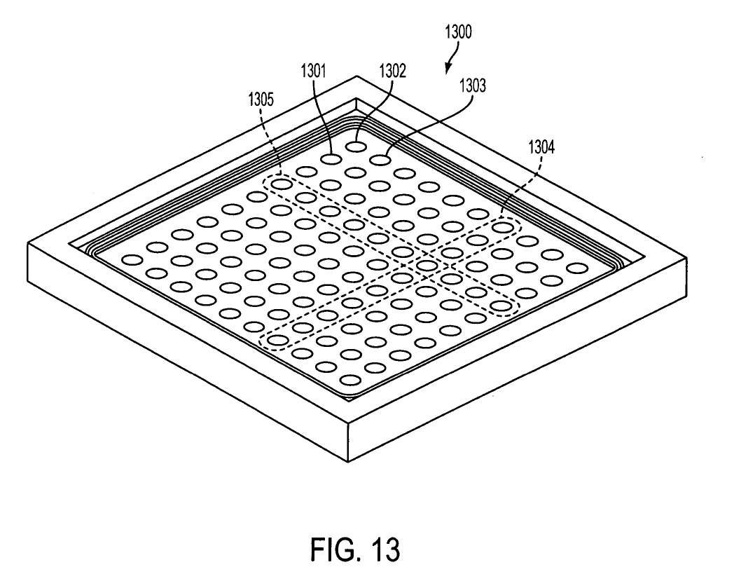

Referring to FIG. 13, an alternative reactor assembly 1300 is shown having a two-dimensional array of reactor wells, such as reactor wells 1301, 1302, and 1303. The reactor wells may be arranged in substantially linear rows, such as row 1304, and columns, such as column 1305, or in any other substantially similar array-like configurations. Where the reactor wells are arranged as shown, each row and/or column may be functionally thought of as performing catalytic chemical reactions that may be measured and/or evaluated by some aspect of the combinatorial spectroscopy device and/or method described in other aspects of the invention. When evaluating catalytic reactions, each reactor well 1301, 1302, 1303, etc. has a first end (e.g., the top) through which reactants may flow into the reactor well, and a second opposing end (e.g., the bottom) through which products of the reaction being analyzed flow. The catalyst(s) may be disposed within each reactor well between the first and second opposing ends. Preferably, the second/bottom end is made of a porous material. The porous material may be any porous material commonly used for catalytic bed surfaces, such as but not limited to metals (e.g., aluminum). The first end is preferably configured such that the optical spectroscopy portions of the system can determine the dynamic bulk and surface nature of the catalytic materials being screened, determining the molecular/electronic structure-activity/selectivity relationship of the catalytic materials or, collecting information on the dynamic structures of the catalytic materials. For example, the first end of each reactor well may be open or may be partially or fully covered by a transparent or semi-transparent material such as diamond, quartz or zinc selenide which enhances IR signals without significantly impeding measurements using Raman or UV-Vis signals

Preferably, the effluent from each reactor well (e.g., 1301, 1302, 1303) is collected for further analysis using chemical spectroscopy (e.g., TPSR). The effluent may be collected in any of a variety of ways well known to those of ordinary skill such that such analysis may be performed. For example, the effluent from each reactor well may be separately collected in a dedicated vessel. This may be desirable where a plurality of reactor wells is analyzed in parallel. Where the reactor wells are analyzed in series, then a single vessel may be used over time to individually collect the effluent from the various reactor wells (and possibly being cleaned in between).

Other aspects of the present invention use the illustrative knowledge-based combinatorial apparatus described herein to create libraries that may significantly reduce the experimental space that will need to be examined and provide molecular structural information of materials for future targeted applications. For example, the libraries may be useful in determining the aging process of a targeted material, usually the key factor in determination of the material's long-term usefulness, and how to best retard the molecular and electronic level changes responsible for the material aging events. The availability of such powerful physical and chemical material characterization instrumentation to the materials community, and especially catalytic materials, will significantly advance the state-of-the-art in new material discovery since the combinatorial libraries may become leveraged in many different materials applications besides the initially targeted application. For example, current combinatorial chemical screening can identify a specific catalytic material for a targeted reaction, but the absence of dynamic bulk and surface information prevents the translation of these materials to other catalytic or non-catalytic material applications. Aspects of the present invention, including the shift to molecular and electronic level investigations has the potential to revolutionize the discovery of new materials, including both crystalline as well as amorphous, and their physical-chemical properties for a wide range of applications including but not limited to catalyst development for novel petroleum, petrochemical, environmental and polymer applications.

Combinatorial libraries may provide organized storage and rapid access to new spectra/data from screening studies. Data can be stored that includes, but is not limited to, the bulk and surface molecular and electronic structures and oxidation states present in the materials being investigated, the chemical characteristics of different catalytic elemental components when appropriate chemical probe molecules are employed as well as kinetic and mechanistic information, and/or the nature of surface species and their coordination characteristics with different cations. Organization, access, searching, and retrieval of information from these libraries can be accomplished using any data storage/access techniques known to those of ordinary skill in the relevant art, including but not limited to database (e.g., using SQL) techniques. The data may be stored on any type of computer-readable media such as but not limited to one or more hard drives, optical and/or magnetic removable disks, magnetic tapes, memories, etc. Such computer-readable media may be readable, writeable, and searchable using one or more computing devices. In some embodiments, standard off-the-shelf database query software may be used (and possibly modified) to access, search or retrieve information based on measurements obtained using other aspects of the present invention. In further embodiment, customized database query software may be created for these purposes. These molecular/electronic structural-based and chemical-based libraries can be used to determine the optimum molecular and electronic properties that will give the best material performance for a specific targeted application. The libraries may be used to compare the findings in order to (1) analyze and interpret the molecular and electronic data; and (2) determine molecular/electronic structure-activity/selectivity relations for the catalytic system; (3) determine reaction kinetics and mechanisms; and (4) guide subsequent combinatorial screening studies of catalytic materials with improved performance employing a knowledge-based approach. In addition, new combinatorial libraries may also be generated for specific catalytic systems that will serve as a guide for future screening studies of different chemical functionalities (e.g., alcohols, ketenes, olefins, alkenes, aromatics, etc.). These combinatorial libraries may become a beneficial component for data analysis and future combinatorial operando spectroscopy reactor screening studies, especially when combined with well-established software engines that rapidly locate the optimal points in a given set of data.

In summary, it has been demonstrated on a non-combinatorial basis, that both operando and chemical spectroscopy protocols facilitate the generation of practical and fundamental information that have not previously been obtained on complex catalytic materials. Using combinatorial techniques to increase the speed of these molecular/electronic structure-surface reactivity techniques will truly revolutionize the discovery process in a wide spectrum of materials applications. In addition, by combining transient kinetic experiments with traditional steady-state measurements, it has been possible to obtain the surface kinetics and reaction mechanisms of complex surface reaction pathways in an unprecedented fashion. By applying the same protocol of transient kinetic experiments with steady-state measurements of catalysts that are in the process of deactivating, it will be possible to develop a molecular/electronic-based kinetic model of the deactivation process. All of these results will be available to the catalytic and materials researchers in days or even hours rather than the months of experimentation as used to be the case.

What is claimed is:

1. An apparatus for material development comprising:
a combinatorial reactor array having chambers, a reaction occurring in each respective chamber;
a first instrument configured to measure bulk and surface structures and surface species during each respective reaction; and
a second instrument configured to measure the reaction products from each chamber during each respective reaction.

2. The apparatus of claim 1, wherein the reaction is a catalytic reaction.

3. The apparatus of claim 1, wherein the first and second instruments each makes their respective measurements as a time series of measurement samples during each respective reaction.

4. The apparatus of claim 2, wherein the first instrument includes two devices that each uses a different technique for measuring the catalyst bulk and surface structures of a given one of the catalysts.

5. The apparatus of claim 3, wherein only one of the two devices is an FT-IR device.

6. The apparatus of claim 1, wherein the first instrument takes measurements using optical spectroscopy and the second instrument takes measurements using spectroscopy other than optical spectroscopy

7. The apparatus of claim 1, wherein the second instrument is a gas chromatograph/mass spectroscopy measurement device.

8. The apparatus of claim 1, wherein the second instrument is a TPSR measurement device.

9. The apparatus of claim 1, wherein the combinatorial reactor array includes a plurality of reactor channels, each reactor channel having a reactor chamber that is at least partially optically transparent.

10. The method of claim 1, wherein the first and second instruments perform their respective measurement at a substantially identical time.

11. The method of claim 2, wherein the catalytic reactions are not quenched prior to the measurements being taken.

12. A method for material development, comprising:
providing a combinatorial reactor array having a plurality of chambers;
causing a reaction to occur in each respective chamber;
measuring bulk and surface structures and surface species during each respective reaction; and
measuring a reaction product from each chamber during each respective reaction.

13. The method of claim 12, wherein the reaction is a catalytic reaction.

14. The method of claim 13 wherein each of the steps of measuring include taking a time series of measurement samples during each respective catalytic reaction.

15. The method of claim 13, wherein the step of measuring the catalyst bulk and surface structures and surface species includes taking a measurement using optical spectroscopy and the step of measuring the reaction product includes taking a measurement using a technique other than optical spectroscopy.

16. The method of claim 12, wherein the step of measuring the reaction product includes making a gas chromatography/mass spectroscopy measurement.

17. The apparatus of claim 12, wherein the step of measuring the reaction product includes making a TPSR measurement.

18. A method for measuring catalyst performance, comprising:
performing combinatorial analysis of products of simultaneous catalytic reactions during the catalytic reactions; and
performing optical spectrographic analysis of catalysts involved in the catalytic reactions during the catalytic reactions.

19. The method of claim 18, wherein the performing steps occur at a substantially identical time.

20. The method of claim 18, wherein the catalytic reactions are not quenched prior to the performing steps.
